(19) United States
(12) Patent Application Publication
(10) Pub. No.: US 2009/0136430 A1
(43) Pub. Date: May 28, 2009

(54) Antihistamine/Corticosteroid preparations for the treatment of atopic dermatitis

(76) Inventor: Harry A. Dugger, Flemington, NJ (US)

Correspondence Address: OMRI M. BEHR, 325 PIERSON AVENUE, EDISON, NJ, 08837-3123, US

(21) Appl. No.: 11/945,842

(22) Filed: Nov. 27, 2007

(51) Int. Cl. A61K 31/56; A61K 9/12; A61P 17/00

(52) U.S. Cl. 424/45; 514/171

(57) ABSTRACT

It has been found that the combination of an antihistamine with a corticosteroid is more effective in the treatment of atopic dermatitis than either one used separately. The synergistic effect in some cases results in the disappearance of the atopic dermatitis lesion within one to five days with little or no relapse. Compositions and the methods of utilizing these preparations are disclosed.

### FIELD OF THE INVENTION

Antihistamine/corticosteroid ointment, foam or spray for the treatment of atopic dermatitis.

### BACKGROUND OF THE INVENTION^{[1a]}

Atopic dermatitis (AD) is a common, Th2 cell-mediated, eczematous skin disorder that affects 15% to 20% of children in developed countries^{[1b]} and 1% to 3% of adults.^{[2]} Epidemiologic studies suggest that there has been a 2- to 3-fold increase in the prevalence of AD during the past 3 decades.^{[3]} The basis for this increased prevalence of AD is poorly understood. In 1989, Strachan^{[4]} postulated the “hygiene hypothesis,” which proposes that allergic diseases, such as AD, might be prevented by “infection in early childhood transmitted by unhygienic contact with older siblings.” Given the fact that IgE-mediated allergic responses are driven by pro-inflammatory cytokines released by Th2 cells, whereas responses to infection are mediated by pro-inflammatory cytokines released by Th1 cells, a decreased number of childhood infections could indeed predispose to enhanced Th2-type allergic responses.^{[5]}

AD is typically a disease of childhood. Most adults who suffer from chronic eczema have had nearly lifelong disease. However, a small percentage of adults (variably estimated at 3% to 5%) may first manifest atopic dermatitis after 18 years of age. Indeed, among studies performed on individuals undergoing patch testing for eczematous disease, approximately 9% of adult patients ≧18 years of age were diagnosed with new-onset atopic dermatitis.^{[6,7]} It is noteworthy that a significant percentage of these adult patients can present with nonflexural involvement and/or dermatitis patterns more akin to nummular dermatitis, prurigo nodularis, or follicular eczema.^{[7,8]} Additionally, a significant number of “burned out” atopic children will present in adulthood with work-related hand eczema, predominantly on the basis of a lowered threshold for irritation. This is particularly true among individuals with an atopic diathesis who enter into “wet work” professions or other professions involving significant exposure to cutaneous irritants.

Given the chronic, relapsing nature of AD and its associated, often intense, pruritus, the disease has a significant impact on the patient's life: social interactions are hindered, self-esteem is lowered, and sleep disturbances are not unusual. In a study using the Dermatology Life Quality Index to assess the effect of severe atopic eczema on the quality of life in 92 adult patients, Finlay^{[10]} noted that adults with severe atopic eczema considered that having diabetes or hypertension would be better than having eczema. Eighty percent of these patients reported that their AD affected family life and 57% reported that the disease hindered sexual relationships.^{[10]} Thirty-two percent of Finlay's patients reported having lost a median of 5000 pounds in income over the prior year because of their eczema.^{[10]} Not surprisingly, 50% of these patients would give up 2 or more hours per day in order to have normal skin, and 74% would pay 1000 pounds or more for a cure.^{[10]}

### DISCUSSION OF THE PRIOR ART

Emollients: traditional treatments for AD have included proper skin care and bathing habits, avoidance of triggers including irritants, and the use of bland emollients. In clinical trials, emollients alone demonstrated enhanced therapeutic responses^{[11-13]}.

Avoidance of food and aeroallergen Triggers: Patients with AD are advised to avoid known triggers. IgE-mediated reactions to foods and/or aeroallergens may exacerbate the disease however IgE-mediated reactions to foods are an uncommon factor in the adult patient with AD.

Glucocorticosteroids

While patients with very mild AD may be controlled by avoiding triggers and maintaining appropriate moisturization, many patients, especially those with more moderate to severe disease, will experience chronic and relapsing flares. Since the 1950s when they were first introduced, topical glucocorticosteroids have been the benchmark therapy for AD.^{[14]} In controlled trials, judicious, intermittent use (twice weekly) of a mid-potency topical steroid has been shown to be safe and efficacious.^{[15,16]}

The issues surrounding local cutaneous side effects from chronic glucocorticosteroid use, including atrophy, striae, telangiectasia, hypopigmentation, and perioral dermatitis, are well known. However, the potential for systemic side effects following long-term, chronic steroid use requires further study. While it is clear that inhaled corticosteroids can result in a significant reduction in bone density; marked suppression of the hypothalamic pituitary axis (HPA) with adrenal suppression; and an enhanced risk for posterior subcapsular cataracts, ocular hypertension, and/or glaucoma,^{[17]} the risks of these side-effects when glucocorticosteroids are used long-term topically has not been extensively studied. The potential for topical steroids to suppress the HPA in pediatric patients has been associated with treatment of patients with more severe disease who are 2 years of age or younger.^{[18-20]} Prolonged use of topical corticosteroids around the eyes has been reported to induce open-angle glaucoma and cataracts,^{[21]} although controlled studies have not been performed. Similarly, there are no studies assessing the impact of long-term treatment with topical steroids on bone mineral density. In the absence of these studies, it would seem well advised to counsel patients undergoing long-term topical steroid therapy to ingest adequate calcium and vitamin D, to undergo surveillance ophthalmic examinations, and to be vigilant regarding signs or symptoms of iatrogenic Cushing's disease.

The Calcineurin Inhibitors

Tacrolimus.

The topical calcineurin inhibitors (tacrolimus and pimecrolimus) work by inhibiting the activation of calcineurin in responding T cells. The safety and efficacy of tacrolimus ointment (Protopic) in reducing the severity of moderate to severe AD in children (≧2 years of age, 0.03% tacrolimus) and in adults (≧16 years of age, 0.1%) has been demonstrated in a number of studies, including those assessing long-term control of the disease.^{[22-25]} In a study of 408 adult atopic dermatitis patients over a 4-year period (median duration of treatment 902 days), no additional adverse events from tacrolimus 0.1% ointment were identified that had not been reported in the 12-week trials.^{[29]}

The comparative efficacy of tacrolimus 0.1% vs topical glucocorticosteroids has been assessed in several studies. Tacrolimus 0.1% was compared to alclometasone dipropionate 0.1% in 143 patients with AD affecting the face and neck; tacrolimus 0.1% was significantly more effective.^{[26]} In another trial comparing tacrolimus 0.03% and 0.1% ointments vs hydrocortisone butyrate 0.1% ointment in 570 adults with moderate to severe atopic dermatitis, tacrolimus 0.1% ointment was as effective, and tacrolimus 0.03% was significantly less effective, than hydrocortisone butyrate 0.1% ointment.^{[28]} Another study^{[27]} compared tacrolimus 0.1% ointment to betamethasone valerate 0.1% ointment in 968 adults with moderate to severe atopic dermatitis of the trunk and extremities and found that tacrolimus was significantly more effective based on the proportion of patients that cleared or achieved excellent improvement.

Pimecrolimus.

A number of studies have likewise evaluated the long-term safety and efficacy of pimecrolimus (Elidel) cream 1%. The most long-term of these studies have been performed in infants and children with moderate to severe disease treated intermittently for up to 2 years.^{[28-30]} In a study of 130 adults with moderate atopic dermatitis who were randomized to receive pimecrolimus cream 1% or vehicle at the first signs/symptoms of AD for a 6-month period, 59.7% of pimecrolimus-treated patients experienced no flares during the study period, compared with 22.1% of vehicle-treated patients.^{[35]} Patients in both treatment groups who experienced a flare of dermatitis were allowed treatment with prednicarbate 0.25% cream. It was noted that corticosteroids were required on significantly fewer days in the pimecrolimus group compared with the vehicle group.^{[31]}

In a similarly designed, randomized trial of 192 adults with moderate to severe atopic dermatitis,^{[32]}44.8% of patients in the pimecrolimus group did not experience a flare, compared with 18.8% of patients in the control group. Furthermore, the median time to first flare was 144 days in the pimecrolimus group and 26 days in the control group.^{[32]} Corticosteroid medication for flares was used on 14.2% of the days during the 24-week treatment period in the pimecrolimus group and in 37.2% of the days in the control group, which represented a significant reduction in the use of steroids by the pimecrolimus group.^{[32]}

To date, the relative potency of pimecrolimus 1% vs topical glucocorticosteroids has been evaluated in 2 studies. In one, a phase 2, double-blind, randomized, parallel-group, multicenter, dose-finding study in 260 patients with moderate to severe AD affecting 5% to 30% of the body surface area, a clear dose-response relationship for pimecrolimus was evident: 0.2%, 0.6%, and the currently marketed 1.0% creams all were significantly more effective than vehicle.^{[33]} When compared to betamethasone valerate 0.1% cream, pimecrolimus 1% was not as effective in treating nonfacial disease during the limited time (3 weeks) of this study.^{[33]} In another randomized, double-blind, multicenter study assessing the long-term safety and tolerability of pimecrolimus cream 1% vs a combination steroid regimen (triamcinolone acetonide 0.1% cream for the trunk and extremities and hydrocortisone acetate 1% cream for face, neck, and intertriginous areas), the combined topical corticosteroid regimen was significantly more effective than pimecrolimus cream 1% after treatment for 1 week, 3 weeks, and 6 months.^{[34]} However, there was no significant difference in outcomes at the end of the 12-month study.^{[34]}

Pimecrolimus vs Tacrolimus.

In a 6-week, randomized, single-blind study of 141 children with moderate atopic dermatitis,^{[35]} pimecrolimus 1% cream was as effective as tacrolimus 0.03% ointment when the proportion of children clear or almost clear at 6 weeks was assessed. With the exception of the results on Day 22 of the study, there was no difference between the treatment groups in the proportion of patients reporting no or mild pruritus.^{[35]} Only one 6-week study has compared tacrolimus 0.1% to pimecrolimus 1% cream in adults.^{[40]} In this randomized, investigator-blinded study of 350 patients≧16 years of age with mild to very severe atopic dermatitis, the median percent reduction in eczema area severity index (EASI) score was significantly greater for the tacrolimus 0.1% ointment (74%) than for pimecrolimus 1% cream (54%).^{[36]}

Safety issues with calcineurin inhibitors. Despite clinical studies (in over 19,000 patients, including more than 7000 children for both pimecrolimus cream [data on file, Novartis Pharmaceuticals] and tacrolimus ointment [data on file, Fujisawa Healthcare]) that showed transient and low systemic exposure and no evidence of systemic immunosuppression for both drugs, the US Food and Drug Administration (FDA) issued a Public Health Advisory dated Mar. 10, 2005,^{[37]} which indicated its intent to require labeling changes for both Elidel (pimecrolimus) cream and Protopic (tacrolimus) ointment that would include placement of a black-box warning about the potential cancer risks of these drugs. This action was based on studies (in mice, rats, and monkeys exposed orally, and in rodents exposed topically) in which cancers developed following exposures to these calcineurin inhibitors at systemic levels more than 25 times higher than the maximum human exposure following topical application. In interpreting these animal data, it must be realized that patients with atopic dermatitis would be expected to have considerably less daily systemic exposure to topical tacrolimus or pimecrolimus when compared to that observed in rodents in lifetime carcinogenicity studies, especially since the skin of the rodent is much more permeable than that of man.^{[38]} Additionally, in the 39-week oral gavage study in cynomolgus monkeys, where pimecrolimus at 15, 45, and 120 mg/kg/day orally was associated with immunosuppression-related lymphoproliferative disorders, it should be kept in mind that the highest blood level observed following topical application of pimecrolimus 1% cream, including in infants as young as 3 months of age with 92% of body surface involvement, was 55 times less than the lowest level with an effect in these monkeys (data on file, Novartis Pharmaceuticals).

The issue of lymphoproliferative diseases in association with systemic (not topical) calcineurin inhibitors has been termed immunosuppression-related post-transplant lymphoproliferative disorders. These disorders constitute a spectrum of B-cell diseases, ranging from benign polyclonal hyperplasia to malignant B-cell lymphomas, which occur in transplant recipients receiving intensive immunosuppressive regimens.^{[39]} Immunosuppressive transplant regimens typically include high doses of calcineurin inhibitors (cyclosporine or tacrolimus systemically), as well as corticosteroids, azathioprine, antilymphocytic antibodies, and other immunosuppressive medications.^{[40,41]} In contrast to lymphomas that occur in immunocompetent individuals, immunosuppressive-related, post-transplant lymphoproliferative disorders are generally treated by a reduction or cessation of the immunosuppressive therapy.^{[42]} Furthermore, a major risk factor for post-transplant lymphoproliferative disease is transplant-related infection with Epstein-Barr virus.^{[39]} In immunocompetent and many immunocompromised individuals, lymphoproliferation does not occur in response to Epstein-Barr virus-transformed B cells due to viral specific cytotoxic T cells and natural killer cells. To date, in clinical studies and postmarketing reports, there have been no cases of B-cell lymphoma or B-cell lymphoproliferative disorders associated with topical use of either tacrolimus ointment or pimecrolimus cream. Furthermore, in clinical studies evaluating both pimecrolimus and tacrolimus applied topically, there has been no evidence that these agents impair dermal or systemic immune function.^{[29,43-45]} Of note, in a 1-year study comparing the safety and efficacy of topical corticosteroids to 1% pimecrolimus cream in adults with moderate to severe atopic dermatitis, individuals treated with topical steroids had a significantly higher ingdence of bacterial infection, especially folliculitis, as well as complicating herpes simplex eruptions.^{[34]}

In murine photocarcinogenicity studies, tacrolimus 0.1% ointment was found to slightly, but significantly, decrease the latency time to onset of first tumor (benign or malignant lesion>1 mm in diameter) when compared with its vehicle (data on file, Fujisawa Healthcare). By contrast, there was no evidence in these studies that either tacrolimus 0.03% (data on file, Fujisawa Healthcare) or pimecrolimus 1% cream (data on file, Novartis Pharmaceuticals) enhanced the rate of photocarcinogenicity relative to their respective vehicles.

In a study by Naylor and colleagues^{[46]} that investigated whether patients in the United States treated with topical tacrolimus ointment had an increased risk for nonmelanoma skin cancer compared with age-specific ingdences reported in the Physician's Health Survey,^{[47]} no evidence of an increased risk for non-melanoma skin cancers was found among the patients treated with tacrolimus ointment. Similarly, there appears to be no evidence for an increased risk of nonmelanoma skin cancers among the more than 5 million patients treated with pimecrolimus 1% cream since it was released in the US market in December 2001 (data on file, Novartis Pharmaceutical Co.).

The lack of clinical evidence of immunosuppression following topical treatment of atopic skin with the calcineurin inhibitors is not surprising, given their molecular size (>800 daltons). Because of their size, the calcineurin inhibitors are unlikely to penetrate intact skin to any significant extent. Furthermore, skin penetration of these molecules will diminish as the inflammatory component of the atopic disease resolves with therapy. By contrast, glucocorticosteroids, with molecular weights<500 daltons, have the ability to penetrate both normal and impaired skin and, as discussed earlier, represent a greater threat to systemic absorption than do the calcineurin inhibitors. It is not surprising that, in a 1-year study of adults with moderate to severe atopic dermatitis treated with tacrolimus ointment, blood levels were consistently found to be low.^{[43]} In this study,^{[47]} 74.7% of patients had no detectable levels of tacrolimus (<1 nanogram/mL) and, in 16.8% of patients, blood levels were between 1 and 2 nanograms/mL. In only 1 patient were levels in the range of a trough level for immunosuppression (5.75 nanograms/mL) and, in this same patient, all subsequent samples during the course of this 1-year study were<1 nanogram/mL.^{[43]} In adult patients with atopic dermatitis treated with pimecrolimus 1% cream, systemic levels have been similarly low, the highest level reported to date being 1.4 nanograms/mL (data on file, Novartis Pharmaceuticals, Inc.).

Thus, given the lack of significant systemic exposure following topical application of these medications, and given the lack of data in humans linking the use of topical calcineurin inhibitors to B-cell lymphoproliferative disorders or cutaneous malignancies, it is not surprising that, in response to the FDA's announcement of its intention to add a black-box warning to the labeling for these agents, the American Academy of Dermatology stated that it was “disappointed that the FDA has taken this action, despite the fact that there is no data that proves proper topical use of pimecrolimus and tacrolimus is dangerous in people.”^{[48]}

Other Topical Therapies

There have been few scientifically controlled trials of topical coal tar in the treatment of atopic dermatitis.^{[49]} Given the cosmetic unacceptability of coal tar, it is unlikely that most patients would comply with treatment. Topical antipruritics should be used cautiously, especially given the potential for the development of allergic contact dermatitis when these agents are applied to inflamed and damaged skin.

Oral Antihistamines

Although sedating and nonsedating antihistamines are commonly used in the management of AD, there is no evidence to support the efficacy of nonsedating antihistamines.^{[50]} Regarding the reported improvements in disease severity and quality of life following treatment with sedating antihistamines, these effects could be driven by enhanced nocturnal sleep, rather than as a result of reduction in symptoms.^{[51,52]} Nonetheless, since many patients with atopic dermatitis have associated rhinoconjunctivitis and/or dermatographism—and given the relative safety profile of oral antihistamines—at least some patients with AD may clearly benefit from oral antihistaminic therapy.

Systemic Therapies

Cyclosporine has been extensively studied in randomized controlled trials for the treatment of severe atopic dermatitis in adults.^{[53-56]} In these trials, patients experienced prompt relief of symptoms; in some cases, the maximal response occurred within 2 weeks of starting treatment.^{[56]} However, following cessation of therapy, the majority of patients relapse within 6 weeks.^{[55]} The renal toxicity and other systemic side effects of cyclosporine limit its long-term usefulness in the treatment of AD.

Glucocorticosteroids, both oral and intramuscular, have been commonly used in the treatment of refractory AD, reportedly with good results.^{[47]} Surprisingly, there have been no randomized controlled trials of systemic glucocorticosteroid therapy in the treatment of AD. Due to side effects (including enhanced photocarcinogenicity with systemic steroids, as reported in a study by Danes and colleagues^{[48]}) and issues related to tachyphylaxis and rebound flares, systemic glucocorticosteroids should be used sparingly and only to quell major flares of AD in adults.

Other systemic therapies for treating AD, including azathioprine, mycophenolate mofetil, methotrexate, intravenous gamma-globulin, and interferon gamma, have some, but very limited, evidence of efficacy.

Ultraviolet Irradiation

Numerous studies in the literature support the role of broad-band UVB,^{[59]} narrow-band UVB,^{[60,61]} PUVA,^{[62]} and high-dose UVA-1 therapy^{[63]} in the treatment of atopic dermatitis. While PUVA clearly increases the subsequent risk of squamous cell carcinoma, the risks of skin cancers associated with UVB and high-intensity UVA-1 appear to be low, on the basis of more than 75 years of data in patients treated with UVA and UVB for psoriasis. Although they are potentially safer alternatives to systemic therapy for adult AD, these modalities do have their limitations. Chief among them are the severe lifestyle restrictions of having to travel to a treatment center multiple times per week. Furthermore, the patient's copayment for these treatments under many managed care plans may range from $15-$30 per treatment, making the costs of therapy prohibitive for some patients.

Systemic and Topical Antibiotics

Many individuals with AD are colonized with Staphylococcus aureus in both affected and nonaffected skin.^{[64,65]} Although oral antibiotics can be beneficial in the presence of impetigo and/or other cutaneous bacterial infections, there is little evidence to support the use of antibiotics, either topically or orally, in clinically uninfected AD.^{[66-68]}

Miscellaneous Therapies

There is little scientific evidence to support the use of dietary supplements, including treatment with fish oil, primrose oil, antioxidant vitamins, or zinc. Herbal supplements have not been found to improve AD in placebo-controlled trials, and concerns about toxicity are an issue. The roles of hypnotherapy/biofeedback and other relaxation techniques have yet to be proven.

### CONCLUSION

The above treatments require long periods of therapy (up to 26 weeks) and are often only palliative in that there is a reduction in symptoms such as itching. Once the treatment is terminated the lesion often remains or returns and the itching returns when the medication wears off. In addition the ingdence of side effects for the more aggressive treatments such as systemic glucocorticoid steroids and the Calcineurin Inhibitors can be so severe that treatment has to be discontinued.

### SUMMARY OF THE INVENTION

The AD irritation is characterized by itching, appearance of red and rough skin. After application of the preparation the itching stops and the redness recedes. The skin returns to its nearly normal condition within 2-5 days. In the cases treated to date the lesion does not return for at least 8-18 months or until exposure to the causative agent reoccurs. In the prior art, weeks of treatment are required and in many cases reduction in the itching occurs but the lesion does not disappear and the itching returns.

Contrary to the above teachings that the symptoms of AD can be controlled by the use of either topical glucocorticoid or oral antihistamines when used individually, it has been found that the topical use of glucocorticoid steroid and an antihistamine in combination at a fixed ratio and in particular when the antihistamine has a high intrinsic activity results in relief of the itching and rapid disappearance of the lesion usually within 2-5 days whereas the prior art required weeks of treatment which often resulted in the appearance of side effects which if severe enough required the discontinuation of treatment.

The invention provides a topically administrable formulation for the treatment of atopoic dermatitis in a mammal affected therewith having active ingredients consisting essentially of at least one antihistamine in its nonionized form or as the pharmaceutically acceptable salt thereof and at least one compound selected from the group consisting of corticosteroids and glucocortico steroids, formulated in a pharmacologically acceptable carrier. Suitably the active ingredients are dissolved in said carrier. The carrier may be a polar solvent and may additionally comprises a non polar solvent, it may then additionally comprise an emulsifying agent.

The formulation of may be administrable as a pump spray, an aerosol spray or a foam. Where appropriate a propellant may also be present. It may also be administrable as an ointment.

It has been found useful that the antihistamine in its nonionized form or as the pharmaceutically acceptable salt thereof salt comprises between about 0.001 and about 5.0 wt % of the entire formulation and the corticosteroid or glucocortico steroid component comprises between about 0.001 and about 5.0 wt % of the entire formulation. Suitably, the antihistamine comprises between about 0.01 and about 5.0 wt % of the entire formulation.

While by no means limited thereto, the antihistamine is suitably selected from the group consisting of clemastine, chlorpheniramine, triprolidine, dextromethorphan, citirizine, fexofenadine, montelukast, diphenhydramine and doxylamine in the nonionized form or as the pharmaceutically acceptable salt thereof. It may also be selected from the group consisting of astemizole, loratadine, and desloratidine in the non-ionized form.

While by no means limited thereto, the corticoid or glucocortico steroid is suitably selected from the group consisting of betamethasone dipropionate or valerate, fluocinonide, alclometasone dipropionate, fluocinolone acetonide, clobetasol propionate, flurandrenolide, monetasone furoate, hydrocortisone butyrate, halobetasol propionate, triamcinolone acetonide, and flucloronide.

The Invention further provides method of treating atopic dermatitis in a mammal afflicted with same, by applying active ingredients consisting essentially of at least one antihistamine in its nonionized form or as the pharmaceutically acceptable salt thereof and at least one corticoid or at least one glucocortico steroid to the afflicted skin areas thereof. The antihistamines and steroids are suitably, but not limited to, those listed above. In this method the active ingredients may be applied as a mixture or substantially contemporaneously.

While by no means limited thereto, the effective antihistamines, most suitably, clemastine or chlorpheniramine in combination with effective corticosteroids most suitably, triamcinolone actonide, fluocinonide, or betamethasone dipropionate can be delivered to the site of the lesion either as an ointment to be massaged into the skin by hand, or as a spray or foam also to be massaged into the skin by hand. If a salt is to be used, a pump spray and polar solvents would be preferred. If the free base should be used as the active ingredient, either a pump spray or foam, or an aerosol spray or foam using a propellant can be employed. After applying the spray, foam or ointment, the resulting fine layer of liquid can be massaged into the skin by hand. The use of the word ointment is intended to included gels and creams.

The only limit on the solvents to be used for the spray, foam or ointment is that they must be compatible with topical applications, and at least one of them should have the properties of a dermal penetration enhancer. These ointments can be packaged in foil packs or in tubes for one time use or in larger containers for multiple uses.

### DESCRIPTION OF THE PREFERRED EMBODIMENTS

Formulation of spays for Topical Application

In the preferred embodiments of the present invention there are provided spray composition for topical administration of antihistaminically active compounds and corticosteroids or a glucocorticoid where these active compounds are soluble in a pharmacologically acceptable polar solvent, the spray comprises, in weight % of total composition: polar solvent 90-99.9%, active compounds 0.002-10%. In some cases a mixture of polar and non-polar solvents can be used as long as a homogenous solution is obtained

Where the active compounds are soluble in a pharmacologically acceptable non-polar solvent the spray composition comprises in weight % of total composition: a pharmaceutically acceptable propellant, 45-93%, non-polar solvent 5-55%, and active compounds 0.002-10%. In some cases a mixture of polar and non-polar solvents can be used as long as the combination is compatible with the propellant such that a homogenous solution is obtained.

While the invention is in no way limited thereto, as active antihistamines clemastine, chlorpheniramine, astemizole, triprolidine, hydroxyzine, dextromethorphan, citirizine and loratadine in their nonionized form or as the pharmaceutically acceptable salts thereof, have been found most suitable.

While the invention is in no way limited thereto, as active corticosteroid or glucocorticoid betamethasone dipropionate or valerate, triamcinolone acetonide, fluocinonide, alclometasone dipropionate, fluocinolone acetonide, clobetasol propionate, flurandrenolide, monetasone furoate, hydrocortisone butyrate, halobetasol propionate have been found most suitable.

Additionally, the topical spray compositions may comprise, by weight of total composition: aromatizing agent 1-10%, suitably synthetic or natural oil of peppermint, oil of spearmint, rose oil, citrus oil, fruit aromas, perfumes and aromas commonly used in ointments and lotions and combinations thereof.

As preferred polar solvents there may be mentioned low molecular weight polyethylene glycols (PEG) of 200-600 MW, C₂₋₈ mono- and polyalcohols, and alcohols of C₇₋₈ hydrocarbons of a linear or branched configuration. Most suitable in this group are polyethylene glycol and ethanol.

As non polar solvents, there may be utilized (C₂- fatty acid(C₂₋₆)esters, (C₇₋₁₈)hydrocarbons of a linear or branched configuration, and (C₂₋₆)alkanoyl esters, and triglycerides of said fatty acids. Most suitably, miglyol.

A combination of polar and non-polar solvents can be used as long as they are compatible with the propellant such that a homogenous solution is obtained at 0-40 degrees C.

There are also provided lotion compositions, including ointments, creams, emulsions and the like, for topical administration of antihistaminically active compounds in combination with a corticosteroid or glucocorticoid such as the general group and preferred species listed above, wherein the composition comprises in weight % of total composition: solvent 75-99.99%, active compounds 0.002-10%.

If desired, the foregoing aromatizing agents may also be used. As solvents, the polar and non polar solvents listed above are also operative.

The occurrence of atopic dermatitis in a mammal may be modified or relieved by administering an antihistaminically pharmacologically active compound in combination with a corticosteroid or glucocorticoid to said mammal having been exposed to a atopic dermatitis causing substance, by spraying the potentially affected skin location thereof with any of the forgoing spray compositions, or applying any of the foregoing lotion compositions.

Drug Substance Properties:

Antihistamines which may be used are limited to those that exhibit antihistamine properties and that are soluble enough in the solvent of choice to lead to solutions having a concentration of 0.001-5% w/w. Suitably, there may be used clemastine, chlorpheniramine, astemazole, triprolidine, dextromethorphan, citirizine and loratadine in their nonionized form or as the pharmaceutically acceptable salts thereof. The antihistamine of choice would be clemastine hydrogen fumarate or clemastine base. A second first choice would be chlorpheniramine hydrochloride or base. Corticosteroids and glucocorticoids which may be used are limited to those that exhibit anti inflammatory and antipruritic properties and that are soluble enough in the solvent of choice to lead to solutions having a concentration of 0.001 to 5.0% w/w. Suitably, there may be used betamethasone dipropionate or valerate, triamcinolone acetonide, fluocinonide, alclometasone dipropionate, fluocinolone acetonide, clobetasol propionate, flurandrenolide, monetasone furoate, hydrocortisone butyrate, halobetasol propionate.

The preferred polar solvent properties are as follows: For salts and such polar drugs, one can use water, low molecular weight alcohol's (preferable ethanol), polyethylene glycols (PEG) in the range 200-600, low molecular weight ketones such as acetone and combinations thereof. At least one of the solvents acts as a dermal penetration enhancer.

The preferred non-polar solvent properties may be expressed as follows: Non-polar, C₇₋₁₈ hydrocarbons and their alcohols, esters of fatty acids, fatty acid triglycerides such as migylol, must be miscible with the propellant such that one phase is formed at temperatures 0-40.degree. C. One of the solvents acts as a dermal penetration enhancer.

In the case of a foam formulation, the composition comprises an oil and water combination emulsified using an emulsifying agent commonly known in the art. Hence both a polar and non-polar solvent will be required.

Optionally, there may be employed aroma agents such as: oil of peppermint, oil of spearmint, oil of wintergreen, citrus oils, both synthetic and natural as well as oil of rose or other perfumes which are normally used in creams and lotions.

A pump spray of the invention comprises the following formulation:

| | | | Most |
|---|---|---|---|
| | Amount | Preferred Amount | preferred amount |
| Polar solvent | 75-99.9% | 85-99.8% | 90-99.4% |
| Corticosteroid | 0.001-5.0% | 0.002-4.0% | 0.01-3.0% |
| Antihistamine Salt | 0.01-5.0% | 0.02-4.0% | 0.1-3.0% |
| Aroma | 0.05-10% | 0.1-8.0% | 0.5-6.0% |

A metered dose valve is preferred so that a measured amount of the preparation is deposited on the site.

Formulation of Foams for Topical Application

Foams can be formulated as is common in the art usually as oil/water emulsions and as such use the above solvents as well as emulsifying agents to maintain the mixture as an emulsion. These foams can be delivered to the site of the atopic dermatitis either in a pump or aerosol delivery system. In either case a metered dose valve is preferred so that a measured amount of the preparation is deposited on the site.

Foams of the invention comprises the following formulation:

| | | Preferred | Most preferred |
|---|---|---|---|
| | Amount | Amount | amount |
| Polar solvent | 40-60% | 30-55% | 35-50% |
| Non-polar solvent | 40-60% | 30-55% | 35-50% |
| Corticosteroid | 0.001-5.0% | 0.002-0.7% | 0.002-3.0% |
| Antihistamine Salt | 0.01-5.0% | 0.02-0.7% | 0.02-3.0% |
| Emulsifying agents | 1-10% | 2-8% | 2.5-5% |
| Aroma | 0.05-10% | 0.1-8.0% | 0.5-6.0% |

Formulation of Ointments for Topical Application

The preferred Solvent Properties for the Solution Formulation are: Solvents such as polyethylene glycols (PEG) of the 200-1000 molecular weight, but preferred are those in the 200-600 range, fatty acid esters and triglycerides. Low molecular weight alcohols and poly-alcohols can also be used. One of the solvents acts as a dermal penetration enhancer. If the ointment or gel is packaged as a soft gelatin capsule, glycerin and water should be used sparingly as they will migrate into the shell and weaken the shell or make it tacky.

Optionally Aroma Agents may be employed such as: Oil of peppermint, oil of spearmint, oil of wintergreen, citrus oils, both synthetic and natural. Oil of rose or other perfumes normally used in creams and lotions may be used.

An ointment of the invention comprises the following formulation:

| | | Preferred | Most preferred |
|---|---|---|---|
| | Amount | Amount | amount |
| Polar solvent | 75-99.99% | 85-99.9% | 90-99.3% |
| Corticosteroid | 0.001-5.0% | 0.002-4.0% | 0.002-3.0% |
| Antihistamine | 0.01-5.0% | 0.02-4.0% | 0.02-3.0% |
| Salt | | | |
| Aroma | 0.05-10% | 0.1-8.0% | 0.5-6.0% |

### EXAMPLES

### Example 1

A specific formulation for clemastine hydrogen fumarate and Triamcinolone acetonide as a topical spray:

| | | |
|---|---|---|
| a. | Clemastine hydrogen fumarate | 0.107 gm |
| b. | Triamcinolone acetonide | 0.04 gm |
| c. | Polypropylene glycol | 11.7 gm |
| d. | Glycerin | 0.58 gm |
| e. | Ethanol | 29.8 gm |
| f. | Water | 1.6 gm |
| g. | Oil of peppermint | 0.78 gm |

1 or 2 activations of 50 micro liters each would be used to deliver a therapeutic amount of the combination to lesion of moderate size (3×6 cm in area)

In accordance with the above formulation, but where, in place of clemastine hydrogen fumarate there is utilized chlorpheniramine maleate, astemizole, triprolidine hydrochloride, dextromethorphan hydrobromide, citirizine hydrochloride or loratadine, and in place of triamcinolone acetonide there is used betamethasone dipropionate or valerate, fluocinonide, alclometasone dipropionate, fluocinolone acetonide, clobetasol propionate, flurandrenolide, monetasone furoate, hydrocortisone butyrate or halobetasol propionate, or fluocinonide, a spray of similar activity is obtained, however actual dosage amounts of the active substance as well as the amounts of solvents will vary.

### Example 2

A specific formulation for clemastine hydrogen fumarate and betamethasone dipropionate as a topical ointment:

| | | |
|---|---|---|
| a. | Clemastine hydrogen fumarate | 0.362 gm |
| b. | Betamethasone dipropionate | 0.270 gm |
| c. | Polypropylene glycol | 46.4 gm |
| d. | Glycerin | 2.30 gm |
| e. | Ethanol | 10.6 gm |
| f. | Water | 0.53 gm |
| g. | Oil of peppermint | 3.08 gm |

1 or 2 drops of approximately 50 micro liters each would be used to deliver a therapeutic amount of the combination to lesion of moderate size (3×6 cm in area)

In accordance with the above formulation, but where, in place of clemastine hydrogen fumarate there is utilized chlorpheniramine, astemizole, triprolidine, dextromethorphan, citirizine and loratadine, and in place of betamethasone dipropionate there is used betamethasone valerate, fluocinonide, alclometasone dipropionate, fluocinolone acetonide, clobetasol propionate, flurandrenolide, monetasone furoate, hydrocortisone butyrate, halobetasol propionate, triamcinolone acetonide, or fluocinonide, a ointment of similar activity is obtained, however actual dosage amounts of the active substance as well as the amounts of solvents will vary.

### Example 3

A specific formulation for clemastine fumarate, Triamcinolone acetonide and betamethasone dipropionate as a topical ointment:

| | | |
|---|---|---|
| a. | Clemastine hydrogen fumarate | 0.362 gm |
| b. | Betamethasone dipropionate | 0.135 gm |
| c. | Triamcinolone acetonide | 0.135 gm |
| d. | Polypropylene glycol | 46.4 gm |
| e. | Glycerin | 2.30 gm |
| f. | Ethanol | 10.6 gm |
| g. | Water | 0.53 gm |
| h. | Oil of peppermint | 3.08 gm |

### Example 4

Treatment of atopic dermatitis with composition of Example 2. On Day 1 subject was diagnosed by his physician as having atopic dermatitis on his back. The physician prescribed a topical corticoid steroid (betamethasone propionate) which the subject applied several times a day to relieve itching. After one week with no improvement in the scope of the lesion or intensity of the itching once the effect of the ointment wore off, the subject applied a topical antihistamine and again the itching was relieved but after a week the lesion was unchanged. On Day 15 the subject applied a combination product of Example 2 to the site and again the itching was relieved and after 4 days of treatment the lesion regressed and the skin returned to a normal condition. There was no relapse as of 18 months.

### REFERENCES

1. a. Belsito D., Management of Atopic Dermatitis in the Adult Patient. Medscape Dermatology 2005; 6 (1). b. Williams II, Robertson C, Stewart A, et al. Worldwide variations in the prevalence of symptoms of atopic eczema in the international study of asthma and allergies in childhood. J Allergy Clin Immunol. 1999;103:125-138.
2. Ashcroft D M, Dimmock P, Garside R, et al. Efficacy and tolerability of topical pimecrolimus and tacrolimus in the treatment of atopic dermatitis: meta-analysis of randomized controlled trials. BMJ. 2005;330:516. Epub February 2005 24.3. Schultz-Larsen F, Hanifin J M. Epidemiology of atopic dermatitis. Immunol Allergy Clin North Am. 2002;22:1-24.
3. Strachan D P. Hay fever, hygiene, and household size. BMJ. 1989;2991259-1260.
4. Romagnani S. The role of lymphocytes in allergic disease. J Allergy Clin Immunol. 2000;105:399-408.
5. Bannister M J, Freeman S. Adult-onset atopic dermatitis. Australas J Dermatol. 2000;41:225-228.
6. Ingordo V, D'Andria G, D'Andria C. Adult-onset atopic dermatitis in a patch test population. Dermatology. 2003;206:197-203
7. Ozkaya E. Adult-onset atopic dermatitis. J Am Acad Dermatol. 2005;52:579-582.
8. Shmunes E. The role of atopy in occupational skin diseases. Occup Med. 1986;1:219-228.
9. Finlay A Y. Measures of the effect of adult severe atopic eczema on quality of life. J Eur Acad Dermatol Venereol. 1996;7:149-154.
10. Hanifin J M, Rajka G. Diagnostic features of atopic dermatitis. Acta Derm Venereol Suppl (Stockh). 1980;92:44-47.
11. Hanifin J M, Hebert A A, Mays S R, et al. Effects of a low-potency corticosteroid lotion plus a moisturizing regimen in the treatment of atopic dermatitis. Curr Ther Res Clin Exp. 1998;59:227-233.
12. Chamlin S L, Frieden I J, Fowler A, et al. Ceramide-dominant, barrier-repair lipids improve childhood atopic dermatitis. Arch Dermatol. 2001;137:1110-1112.
13. Lever R, MacDonald C, Waugh P, Aitchison T. Randomized controlled trial of advice on an egg exclusion diet in young children with atopic eczema and sensitivity to eggs. Pediatr Allergy Immunol. 1998;9:13-19.
14. Van Der Meer J B, Glazenburg E J, Mulder P G, et al. The management of moderate to severe atopic dermatitis in adults with topical fluticasone propionate: The Netherlands Adult Atopic Dermatitis Study Group. Br J Dermatol.1999; 140:1114-1121.
15. Hanifin J T, Gupta A K, Rajagopalan R. Intermittent dosing of fluticasone propionate cream for reducing the risk of relapse in atopic dermatitis patients. Br J Dermatol. 2002;147:528-537.
16. Lipworth B J. Systemic adverse effects of inhaled corticosteroid therapy: a systematic review. Arch Intern Med. 1999;159:941-955.
17. Matsuda K, Katsunuma T, Likura Y, et al. Adrenocortical function in patients with severe atopic dermatitis. Ann Allergy Asthma Immunol. 2000;85:35-39.
18. Tan M H, Lebwohl M, Esser A C, et al. The penetration of 0.005% fluticasone propionate ointment in eyelid skin. J Am Acad Dermatol. 2001;45:392-396.
19. Friedlander S F, Hebert A A, Allen D B. Safety of fluticasone propionate cream 0.05% for the treatment of severe and extensive atopic dermatitis in children as young as 3 months. J Am Acad Dermatol. 2002;46:387-393.
20. Fisher D A. Adverse effects of topical corticosteroid use. West J Med. 1995;162:123-126.
21. Koo J, Prose N, Fleischer A, et al. Safety and efficacy of tacrolimus ointment monotherapy in over 7,900 atopic dermatitis patients: results of an open-label study [abstract]. Ann Dermatol Venereol. 2002;129:1S414.
22. Kang S, Lucky A W, Pariser D, et al. Long-term safety and efficacy of tacrolimus ointment for the treatment of atopic dermatitis in children. J Am Acad Dermatol. 2001;44(suppl):S58-S64
23. Reitamo S, Rustin M, Ruzicka T, et al. Efficacy and safety of tacrolimus ointment compared with that of hydrocortisone butyrate ointment in adult patients with atopic dermatitis. J Allergy Clin Immunol. 2002;109:547-555.
24. Paller A; and the Tacrolimus Ointment Study Group. Long-term safety and efficacy of tacrolimus ointment monotherapy in atopic dermatitis patients: open-label study results [abstract]. Ann Dermatol Venereol. 2002;129:1S247.
25. Nakagawa II. Comparative study of FK506 (tacrolimus) ointment vs alclometasone dipropionate ointment in atopic dermatitis (face and neck lesions) [abstract]. J Invest Dermatol. 1998;110:683. 31. FK506 Ointment Study Group. Phase III comparative study of FK506 ointment vs betamethasone valerate ointment in atopic dermatitis (trunk/extremities) [in Japanese]. Nishimihon J Dermatol. 1997;59:870-879.
26. Papp K A, Breuer K, Meurer M, et al. Long-term treatment of atopic dermatitis with pimecrolimus cream 1% in infants does not interfere with the development of protective antibodies after vaccination. J Am Acad Dermatol. 2005;52:247-253.
27. Papp K A, Werfel T, Folster-Holst R, et al. Long-term control of atopic dermatitis with pimecrolimus cream 1% in infants and young children: a two-year study. J Am Acad Dermatol. 2005;52:240-246.
28. Papp K, Staab D, Harper J, et al. Effect of pimecrolimus cream 1% on the long-term course of pediatric atopic dermatitis. Int J Dermatol. 2004;43:978-983.
29. Meurer M, Fartasch M, Albrecht G, et al. Long-term efficacy and safety of pimecrolimus cream 1% in adults with moderate atopic dermatitis. Dermatology. 2004;208:365-372.
30. Meurer M, Folster-Holst R, Wozel G, et al. Pimecrolimus cream in the long-term management of atopic dermatitis in adults: a six-month study. Dermatology. 2005;205:271-277.
31. Luger T, Van Leent E J, Graeber M, et al. SDZ ASM 981: an emerging safe and effective treatment for atopic dermatitis. Br J Dermatol. 2001;144:788-794.
32. Luger T A, Lahfa M, Folster-Holst, R, et al. Long-term safety and tolerability of pimecrolimus cream 1% and topical corticosteroids in adults with moderate to severe atopic dermatitis. J Dermatol Treat. 2004;15:169-178.
33. Kempers S, Boguniewicz M, Carter E, et al. Comparison of pimecrolimus cream 1% and tacrolimus ointment 0.03% in pediatric patients with atopic eczema. J EurAcad Dermatol Venereol. 2003;17:32-41.
34. Fleischer A B Jr. Tacrolimus ointment demonstrates superior efficacy over pimecrolimus cream in the treatment of atopic dermatitis. Dermatology Express Report: Expert Commentary. Apr. 1, 2004. Available at: http://www.webhealthsearch.com/reports.asp?idreport=237&fn=545-469-0-04-e&label=Dermatoloy.
35. FDA Public Health Advisory. Elidel (pimecrolimus) cream and Protopic (tacrolimus) ointment. Available at: http://www.fda.gov/cder/drug/advisory/elidel_protopic.htm.
36. Reifenrath W G, Chellquist E M, Shipwash E A, et al. Percutaneous penetration in the hairless dog, weanling pig and grafted athymic nude mouse: evaluation of models for predicting skin penetration in man. Br J Dermatol. 1984;111(suppl 27):123-135.
37. Andreone P, Gramenzi A, Lorenzini, S, et al. Posttransplantation lymphoproliferative disorders. Arch Intern Med. 2003;163:1997-2004.
38. Melosky B, Karim M, Chui A, et al. Lymphoproliferative disorders after renal transplantation in patients receiving triple or quadruple immunosuppression. J Am Soc Nephrol. 1992;2(suppl 12):S290-294.
39. Wilkinson A H, Smith J L, Hunsicker L G, et al. Increased frequency of posttransplant lymphomas in patients treated with cyclosporine, azathioprine, and prednisone. Transplantation. 1989;47:293-296.
40. Penn I. Neoplastic complications of organ transplantation. In: Ginns L C, Cosimi A B, Morris P J, eds. Transplantation. Malden, Mass: Blackwell Science; 1999: 770-786.
41. Reitamo S, Wollenberg A, Schöpf E, et al. Safety and efficacy of 1 year of tacrolimus ointment monotherapy in adults with atopic dermatitis. Arch Dermatol. 136:999-1006
42. Fleisher A B, Ling M, Eichenfield L, et al. Tacrolimus ointment for the treatment of atopic dermatitis is not associated with an increase in cutaneous infections. J Am Acad Dermatol. 2002;47:562-570.
43. Wahn U, Bos J D, Goodfield M, et al. Efficacy and safety of pimecrolimus cream in the long-term management of atopic dermatitis in children. Pediatrics. 110(1 Pt 1):e2, 2002. Available at: http/pediatrics.aappublications.org/cgi/content/full/110/1/e2.
44. Naylor M, Elmets C A, Rico M J, et al. Treatment with topical tacrolimus is not associated with an increase in non-melanoma skin cancers. Poster presented at the 60th Annual Meeting of the American Academy of Dermatology; Feb. 22-27, 2002; New Orleans, La.
45. Frieling U M, Schaumberg D A, Kupper T S, et al. A randomized, 12-year primary-prevention trial of beta carotene supplementation for nonmelanoma skin cancer in the physician's health study. Arch Dermatol. 2000;136:179-184.
46. Press release. American Academy of Dermatology issues statement in response to FDA decision related to two eczema medications. Available at: http://www.aad.org/public/News/NewsReleases/fda_decision.htm.
47. Munkvad M. A comparative trial of Clinitar versus hydrocortisone cream in the treatment of atopic eczema. Br J Dermatol. 1989;121:763-766.
48. Klein P A, Clark R A. An evidence-based review of the efficacy of antihistamines in relieving pruritus in atopic dermatitis. Arch Dermatol. 1999;135:1522-1525.
49. Wahlgren C F, Hagermark O, Bergstrom R. The antipruritic effect of a sedative and a non-sedative antihistamine in atopic dermatitis. Br J Dermatol. 1990;122:545-551.
50. Monroe E W. Relative efficacy and safety of loratadine, hydroxyzine, and placebo in chronic idiopathic urticaria and atopic dermatitis. Clin Ther. 1992;14:17-21.
51. Sowden J M, Berth-Jones J, Ross J S, et al. Double-blind, controlled, crossover study of cyclosporin in adults with severe refractory atopic dermatitis. Lancet. 1991;338:137-140.
52. Salek M S, Finlay A Y, Luscombe D K, et al. Cyclosporin greatly improves the quality of life of adults with severe atopic dermatitis. A randomized, double-blind, placebo-controlled trial. Br J Dermatol. 1993;129:422-430.
53. Granlund H, Erkko P, Sinisalo M, Reitamo S. Cyclosporin in atopic dermatitis: time to relapse and effect of intermittent therapy. Br J Dermatol. 1995;132:106-112.
54. Berth-Jones J, Graham-Brown R A, Marks R, et al. Long-term efficacy and safety of cyclosporin in severe adult atopic dermatitis. Br J Dermatol. 1997;136:76-81.
55. Sidbury R, Hanifin J M. Systemic therapy of atopic dermatitis. Clin Exp Dermatol. 2000;25:559-566.
56. Daynes R A, Harris C C, Conner R J, Eichwald E S. Skin cancer development in mice exposed chronically to immunosuppressive agents. J Natl Cancer Inst. 1979;62:1075-1081.
57. Jekler J, Larkö O. UVA solarium versus UVB phototherapy of atopic dermatitis: a paired-comparison study. Br J Dermatol. 1991;125:569-572.
58. George S A, Bilsland D J, Johnson B E, Ferguson J. Narrow-band (TL-01) UVB air-conditioned phototherapy for chronic severe adult atopic dermatitis. Br J Dermatol. 1993;128:49-56.
59. Reynolds N J, Franklin V, Gray J C, et al. Narrow-band ultraviolet B and broad-band ultraviolet A phototherapy in adult atopic eczema: a randomized controlled trial. Lancet. 2001;357:2012-2016.
60. Sheehan M P, Atherton D J, Norris P, et al. Oral psoralen photochemotherapy in severe childhood atopic eczema: an update. Br J Dermatol. 1993;129:431-436.
61. Krutmann J, Diepgen T L, Luger T A, et al. High-dose UVA therapy for atopic dermatitis: results of a multicenter trial. J Am Acad Dermatol. 1998;38:589-593.
62. Leyden J J, Marples R R, Kligman A M. Staphylococcus aureus in the lesions of atopic dermatitis. Br J Dermatol. 1974;90:525-530.
63. Leung D Y M. Role of Staphylococcus aureus in atopic dermatitis. In: Bieber T, Leung D Y M, eds. New York, N.Y.: Marcel Dekker; 2002:401-418.
64. Ewing C I, Ashcroft C, Gibbs A C, et al. Flucloxacillin in the treatment of atopic dermatitis. Br J Dermatol. 1998;138:1022-1029.
65. Boguniewicz M. Sampson H, Leung S B, et al. Effects of cefuroxime axetil on Staphylococcus aureus colonization and superantigen production in atopic dermatitis. J Allergy Clin Immunol. 2001;108:651-652.
66. Lever R, Hadley K, Downey D, Mackie R. Staphylococcal colonization in atopic dermatitis and the effect of topical mupirocin therapy. Br J Derm. 1998;119:189-198.
67. Belsito, D V, Flotte, T J, Lim, H W, et al. Effect of glucocorticoids on epidermal Langerhans cells. J Exp Med. 1982;155:291-302.
68. Hoetzenecker W, Meingassner J G, Ecker R, et al. Corticosteroids but not pimecrolimus affect viability, maturation and immune function of murine epidermal Langerhans cells. J Invest Dermatol. 2004;122:673-684.
69.

1. A topically administrable formulation for the sustained remission of atopical dermatitis in a mammal affected therewith until re-exposure to the causative agent, having active ingredients consisting essentially of at least one antihistamine in its nonionized form or as the pharmaceutically acceptable salt thereof and at least one compound selected from the group consisting of corticosteroids and glucocortico steroids, dissolved in a pharmacologically acceptable carrier.

2. (canceled)

3. The formulation of claim 1 wherein said carrier is a polar solvent.

4. The formulation of claim 3 wherein the carrier additionally comprises a non polar solvent in the presence of said polar solvent.

5. The formulation of claim 4 additionally comprising an emulsifying agent.

6. The formulation of claim 4 additionally comprising a propellant.

7. The formulation of claim 5 additionally comprising a propellant.

8. The formulation of claim 1 wherein the formulation is administrable as a pump spray.

9. The formulation of claim 4 wherein the formulation is administrable as an aerosol spray.

10. The formulation of claim 5 wherein the formulation is administrable as a foam.

11. The formulation of claim 10 additionally comprising a propellant.

12. The formulation of claim 1 wherein the formulation is administrable as an ointment.

13. The formulation of claim 1 wherein the antihistamine in its nonionized form or as the pharmaceutically acceptable salt thereof salt comprises between about 0.001 and about 5.0 wt % of the entire formulation and the corticosteroid or glucocortico steroid component comprises between about 0.01 and about 5.0 wt % of the entire formulation.

14. The formulation of claim 13 wherein the antihistamine comprises between about 0.02 and about 3.0 wt % of the entire formulation and the corticosteroid or glucocortico steroid component comprises between about 0.002 and about 3.0 wt % of the entire formulation.

15. The formulation of claim 1 wherein the antihistamine is selected from the group consisting of clemastine, chlorpheniramine, triprolidine, dextromorphan, cetirizine, fexofenadine, promethazine, montelukast, dipheniramine and doxylamine said antihistamines being in the nonionized form or as the pharmaceutically acceptable salt thereof.

16. The formulation of claim 1 wherein the antihistamine is selected from the group consisting of astemizole, loratadine, and desloratidine said antihistamines being in the non ionized form.

17. The formulation of claim 1 wherein the corticoid or glucocortico steroid is selected from the group consisting of triamcinolone acetonide, betamethasone dipropionate or valerate, fluocinonide, alclometasone dipropionate, fluocinolone acetonide, clobetasol propionate, flurandrenolide, mometasone furoate, hydrocortisone butyrate, and halobetasol propionate.

18. The formulation of claim 17 wherein the antihistamine is selected from the group consisting of clemastine, chlorpheniramine, triprolidine, dextromorphan, cetirizine, fexofenadine, montelukast, dipheniramine and doxylamine, said antihistamines being in the nonionized form or as the pharmaceutically acceptable salt thereof.

19. The formulation of claim 17 wherein the antihistamine is selected from the group consisting of astemizole, loratadine, and desloratidine said antihistamines being in the non ionized form.

20. A method of providing sustained remission of atopic dermatitis in a mammal afflicted with same until re-exposure to the causative agent, by applying a solution in a carrier of active ingredients consisting essentially of at least one antihistamine in its nonionized form or as the pharmaceutically acceptable salt thereof and a compound selected from the group consisting of at least one corticoid and at least one glucocortico steroid to the afflicted skin areas thereof.

21. The method of claim 20 wherein the active ingredients are applied as a mixture.

22. The method of claim 20 wherein the active ingredients are applied substantially contemporaneously.

23. The method of claim 20 wherein the active ingredients are applied via a metered dose valve.

24. The method of claim 20 wherein said carrier is a non-polar solvent.

25. The method of claim 20 wherein said carrier is a polar solvent.

26. The method of claim 25 wherein the carrier additionally comprises a non polar solvent in the presence of said polar solvent.

27. The method of claim 20 wherein the carrier additionally comprises an emulsifying agent.

28. The method of claim 20 wherein said formulation additionally comprises a propellant.

29. The method of claim 26 wherein said formulation additionally comprises a propellant.

30. The method of claim 23 wherein the formulation is administered as a pump spray.

31. The method of claim 26 wherein the formulation is administered as an aerosol spray.

32. The method of claim 27 wherein the formulation is administered as a foam.

33. The method of claim 27 additionally comprising a propellant.

34. The method of claim 22 wherein the formulation is administered as an ointment.

35. The method of claim 20 wherein the antihistamine in its nonionized form or as the pharmaceutically acceptable salt thereof salt comprises between about 0.001 and about 5.0 wt % of the entire formulation and the corticosteroid or glucocortico steroid component comprises between about 0.01 and about 5.0 wt % of the entire formulation.

36. The method of claim 35 wherein the antihistamine comprises between about 0.02 and about 3.0 wt % of the entire formulation and the corticosteroid or glucocortico steroid component comprises between about 0.002 and about 3.0 wt % of the entire formulation.

37. The method of claim 20 wherein the antihistamine is selected from the group consisting of clemastine, chlorpheniramine, triprolidine, dextromorphan, cetirizine, fexofenadine, promethazine, montelukast, dipheniramine and doxylamine said antihistamines being in the nonionized form or as the pharmaceutically acceptable salt thereof.

38. The method of claim 20 wherein the antihistamine is selected from the group consisting of astemizole, loratadine, and desloratidine said antihistamines said antihistamines being in the non ionized form.

39. The method of claim 20 wherein the corticoid or glucocortico steroid is selected from the group consisting of triamcinolone actinide, betamethasone dipropionate or valerate, fluocinonide, alclometasone dipropionate, fluocinolone acetonide, clobetasol propionate, flurandrenolide, mometasone furoate, hydrocortisone butyrate, halobetasol propionate.

40. The method of claim 39 wherein the antihistamine is selected from the group consisting of clemastine, chlorpheniramine, triprolidine, dextromorphan, cetirizine, fexofenadine, montelukast, dipheniramine and doxylamine said antihistamines being in the nonionized form or as the pharmaceutically acceptable salt thereof.

41. The method of claim 39 wherein the antihistamine is selected from the group consisting of astemizole, loratadine, and desloratidine said antihistamines said antihistamines being in the non ionized form.
